(19) United States
(12) Patent Application Publication
(10) Pub. No.: US 2022/0304563 A1
(43) Pub. Date: Sep. 29, 2022

(54) LENS CLEANING SYSTEM AND METHOD FOR A SURGICAL CAMERA

(71) Applicant: Vicarious Surgical Inc., Waltham, MA (US)

(72) Inventors: Adam Sachs, Waltham, MA (US); Sammy Khalifa, Waltham, MA (US); Marshall Wentworth, Waltham, MA (US); Justin Keenan, Lexington, MA (US); Rolando Rodarte, Cambridge, MA (US)

(21) Appl. No.: 17/704,930

(22) Filed: Mar. 25, 2022

Related U.S. Application Data
(60) Provisional application No. 63/166,587, filed on Mar. 26, 2021.

(51) Int. Cl. A61B 1/12; H04N 5/225; A61B 90/70; B08B 1/00

(52) U.S. Cl. CPC A61B 1/126; H04N 5/22521; A61B 90/70; B08B 1/005; H04N 2005/2255; A61B 2090/701

(57) ABSTRACT

The present disclosure provides systems and methods for cleaning a lens of an imaging device during a surgical procedure within an individual. The imaging device comprises a lens, and the wiping object is positioned a distance away from the imaging device that is configured to contact and move along a path of the device in order to clean it. The present disclosure provides methods for initiating automated cleaning of a lens during a procedure comprising using an eye tracking device fitted and coupled to an individual that may track the movements of the individual to actuate cleaning.

### CROSS-REFERENCE

This application claims the benefit of U.S. Provisional Application No. 63/166,587, filed Mar. 26, 2021, which is entirely incorporated herein by reference.

### BACKGROUND

Numerous surgical procedures utilize cameras as well as scopes to visualize anatomical structures. A lens of a camera or an endoscope may become dirty or obscured during a procedure.

### SUMMARY

Described herein are systems and methods for cleaning a lens or a window of a camera or an endoscope that is placed within a body of an individual during a medical or surgical procedure, and wherein the cleaning of the lens or window occurs in a manner that does not require removal of the camera or endoscope from the body of the individual.

Provided herein are systems for cleaning a lens or window of an imaging device during a surgical procedure within a patient, the system comprising: the imaging device comprising the lens or the window; and a wiping object positioned an offset distance away from the imaging device and configured to contact at least a portion of the lens or the window as the imaging device is moved along a path that uses one or more degrees of freedom of the imaging device. In some embodiments, the imaging device comprises a camera or an endoscope. In some embodiments, contacting the wiping object to the portion of the lens or the window and moving the wiping object relative to the portion of the lens removes a material from at least a portion of a surface of the lens or the window. In some embodiments, the material comprises blood, adipose tissue, body fluid, irrigation, condensation, or a combination thereof. In some embodiments, the moving of the wiping object relative to the portion of the lens or the window comprises moving the imaging device relative to the wiping object. In some embodiments, the moving of the imaging device comprises rotation, translation, or a combination thereof. In some embodiments, the moving of the wiping object relative to the portion of the lens or the window comprises the wiping object being stationary. In some embodiments, the moving of the wiping object relative to the portion of the lens or the window comprises moving the wiping object relative to the imaging device. In some embodiments, the imaging device moves relative to the wiping object at a speed of about 0.5 mm/s to about 10.0 mm/s. In some embodiments, the wiping object and the imaging device are not in contact when the lens or the window cleaning is not occurring. In some embodiments, the body of the imaging device is positioned a distance from the wiping object. In some embodiments, the distance is at least about 0.1 mm to about 1.0 mm. In some embodiments, the wiping object does not cover at least a portion of the lens or the window of the imaging device during the medical or the surgical procedure. In some embodiments, the imaging device comprises a first lens and a second lens, wherein the first lens is positioned behind a first window and the second lens is positioned behind a second window. In some embodiments, the wiping object is configured to contact at least a portion of the first window and at least a portion of the second window sequentially. In some embodiments, the system comprises an additional wiping object, wherein the wiping object is configured to contact at least a portion of the first window and the additional wiping object is configured to contact at least a portion of the second window. In some embodiments, the wiping object is configured to contact at least a portion of the first window and the additional wiping object is configured to contact at least a portion of the second window simultaneously. In some embodiments, the wiping object comprises a pad, a sheet, or a wiper, or a combination thereof. In some embodiments, the wiping object is sized to reduce drag across the surface of the lens or the window when the wiping object is moved relative to the lens. In some embodiments, the wiping object comprises a material treated to increase lubriciousness. In some embodiments, the wiping object comprises a material treated to increase resistance to fluid ingress. In some embodiments, the wiping object comprises a flexible material. In some embodiments, the flexible material molds to the surface of the lens or the window when the wiping object is in contact with the lens or the window. In some embodiments, the wiping object comprises a material to improve surface contact to the surface of the lens or the window. In some embodiments, the wiping object comprises a material compatible with one or more sterilization methods. In some embodiments, the wiping object comprises a substantially non-porous surface. In some embodiments, the wiping object comprises a fluoropolymer. The In some embodiments, the fluoropolymer comprises expanded polytetrafluoroethylene (ePTFE). In some embodiments, the wiping object comprises one or more layers of ePTFE. In some embodiments, the wiping object comprises an inner layer comprising a material having a low compressive modulus and an outer layer of fluoropolymer. In some embodiments, the lens comprises a stack of two or more lenses. In some embodiments, the stack of lenses is about 1 mm thick to about 10 mm thick. In some embodiments, the lens or the window comprises glass, plastic, or a combination thereof. In some embodiments, the lens or the window comprises a flexible material. In some embodiments, the lens or the window comprises a coating. In some embodiments, the coating laminates the lens or the window. In some embodiments, the coating provides an increase in hydrophobicity, hydrophilicity, oleophilicity, anti-fogging, anti-reflection, scratch-resistance, or a combination thereof. In some embodiments, the coating is applied to the lens or the window through spraying, spin coating, chemical vapor deposition, physical vapor deposition, or dipping.

Described herein are methods for cleaning a lens or a window of an imaging device during a medical or surgical procedure in a body of an individual, the method comprising: (a) providing the imaging device to be inserted in the body of the individual, wherein the imaging device is coupled to a wiping object; and (b) contacting and moving the imaging device relative to the wiping object, wherein the contact and movement cleans the lens or the window of the imaging device. In some embodiments, the imaging device comprises a camera or an endoscope. In some embodiments, contacting the wiping object to the portion of the lens or the window and moving the wiping object relative to the portion of the lens removes a material from at least a portion of a surface of the lens. In some embodiments, the material comprises blood, adipose tissue, body fluid, irrigation, condensation, or a combination thereof. In some embodiments, the moving of the wiping object relative to the portion of the lens or the window comprises moving the imaging device relative to the wiping object. In some embodiments, the moving of the imaging device comprises rotation, translation, or a combination thereof. In some embodiments, the moving of the wiping object relative to the portion of the lens or the window comprises the wiping object being stationary. In some embodiments, the moving of the wiping object relative to the portion of the lens or the window comprises moving the wiping object relative to the imaging device. In some embodiments, the imaging device moves relative to the wiping object at a speed of about 0.5 mm/s to about 10.0 mm/s. In some embodiments, the wiping object and the imaging device are not in contact when lens or the window cleaning is not occurring. In some embodiments, the body of the imaging device is positioned a distance from the wiping object. In some embodiments, the distance is at least about 0.1 mm to about 1.0 mm. In some embodiments, the wiping object does not cover at least a portion of the lens or the window of the imaging device during the medical or the surgical procedure to allow for visualization by the imaging device. In some embodiments, the imaging device comprises a first lens and a second lens, wherein the first lens is positioned behind a first window and the second lens is positioned behind a second window. In some embodiments, the wiping object is configured to contact at least a portion of the first window and at least a portion of the second window sequentially. In some embodiments, the system comprises an additional wiping object, wherein the wiping object is configured to contact at least a portion of the first window and the additional wiping object is configured to contact at least a portion of the second window. In some embodiments, the wiping object is configured to contact at least a portion of the first window and the additional wiping object is configured to contact at least a portion of the second window simultaneously. In some embodiments, the wiping object comprises a pad, a sheet, or a wiper, or a combination thereof. In some embodiments, the wiping object is sized to reduce drag across the surface of the lens or the window when the wiping object is moved relative to the lens or the window. In some embodiments, the wiping object comprises a material treated to increase lubriciousness. In some embodiments, the wiping object comprises a material treated to increase resistance to fluid ingress. In some embodiments, the wiping object comprises a flexible material. In some embodiments, the flexible material molds to the surface of the lens or the window when the wiping object is in contact with the lens or the window. In some embodiments, the wiping object comprises a material to improve surface contact to the surface of the lens or the window. In some embodiments, the wiping object comprises a material compatible with one or more sterilization methods. In some embodiments, the wiping object comprises a substantially non-porous surface. In some embodiments, the wiping object comprises a fluoropolymer. In some embodiments, the fluoropolymer comprises expanded polytetrafluoroethylene (ePTFE). In some embodiments, the wiping object comprises one or more layers of ePTFE. In some embodiments, the wiping object comprises an inner layer comprising a material having a low compressive modulus and an outer layer of fluoropolymer. In some embodiments, the lens comprises a stack of two or more lenses. In some embodiments, the stack of two or more lenses is about 1 mm thick to about 10 mm thick. In some embodiments, the lens or the window comprises glass, plastic, or a combination thereof. In some embodiments, the lens or the window comprises a flexible material. In some embodiments, the lens or the window comprises a coating. In some embodiments, the coating laminates the lens or the window. In some embodiments, the coating provides an increase in hydrophobicity, hydrophilicity, oleophilicity, anti-fogging, anti-reflection, scratch-resistance, or a combination thereof. In some embodiments, the coating is applied to the lens or the window through spraying, spin coating, chemical vapor deposition, physical vapor deposition, or dipping. In some embodiments, the moving the imaging device relative to the wiping object wipes the lens or the window at least two times. In some embodiments, the moving imaging device relative to the wiping object wipes the lens or the window at least 5 times. In some embodiments, the method further comprises, subsequent to (b), recording an image score.

Provided herein are methods for initiating automated cleaning of a lens or a window of an imaging device during a medical or surgical procedure, the method comprising: (a) providing an eye tracking device, wherein the eye tracking device comprises an eye tracking hardware; (b) coupling the eye tracking device with at least one eye of an individual performing the medical or the surgical procedure; (c) providing the imaging device to be inserted in the body of the individual, wherein the imaging device is coupled to a wiping object; (d) tracking movements of the individual; and (e) detecting an activating movement among the movements to trigger the imaging device to contract and move relative to a wiping object, wherein the contact and movement cleans the lens or the window of the imaging device. In some embodiments, the movement comprises movement of the eye. In some embodiments, the movement of the eye comprises blinking. In some embodiments, the blinking comprises one blink, wherein the one blink lasts for about 0.3 seconds. In some embodiments, the movement of the eye comprises focusing on a target object or location in space for a period of time. In some embodiments, the period of time is about 2 seconds. In some embodiments, the movement of the eye comprises looking in a first direction followed by looking in a second direction. In some embodiments, the looking in the first direction followed by looking in the second direction occurs within about 1 second. In some embodiments, the movement of the eye comprises a sharp look in a specified direction. In some embodiments, the specified direction is towards a camera coupled to the eye tracking device. In some embodiments, the eye tracking hardware comprises foveal tracking, computer vision, electrical potential measurement, contact lenses, or a combination thereof. In some embodiments, the eye tracking hardware comprises foveal tracking. In some embodiments, the eye tracking device is worn on a head of the individual. In some embodiments, the eye tracking device is positioned a distance from the individual, and wherein a camera tracks the at least one movement. In some embodiments, the eye tracking device is positioned within a stationary viewfinder. In some embodiments, the stationary viewfinder is a stereoscopic microscope.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

Further, headings provided herein are for convenience only and do not interpret the scope or meaning of the claimed disclosure. The section headings used herein are for organizational purposes only and are not to be construed as limiting the subject matter described.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a camera in a front facing configuration along with a wiping object, as described in some embodiments.

FIG. 2 shows a camera in an orthogonal direction to insertion, as described in some embodiments. In some embodiments, a maneuver may be performed first in order to get the camera aligned with the wiping object.

FIG. 3 shows a camera pointed downward, as described in some embodiments. In some embodiments, the camera may move from the previous position past this position so that the entire surface of the lens is cleaned.

FIG. 4 shows a close up of a window and a wiping object, as described in some embodiments.

FIG. 5 shows a surgeon sitting at a control console operating a surgical robot, as described in some embodiments.

FIG. 6 shows a camera with an automated wiper built in, as described in some embodiments.

FIG. 7 shows one example of a computer system that is programmed or configured to direct operation of a device or system as described herein.

### DETAILED DESCRIPTION

Described herein are systems and methods for cleaning a lens or a window of a camera or an endoscope that is placed within a body of an individual during a medical or surgical procedure, and wherein the cleaning of the lens or window occurs in a manner that does not require removal of the camera or endoscope from the body of the individual. Cameras and endoscopes used during medical or surgical procedures may have material build up on a lens of said camera or endoscope which results in an obstruction of the view displayed by the camera or endoscope. Examples of materials that may build up on a lens of a camera or endoscope during a medical or a surgical procedure include but are not limited to dirt, blood, adipose tissue, body fluid, irrigation, condensation, or a combination thereof.

The traditional approach to cleaning a lens of a camera or endoscope used in a medical or surgical procedure is to remove the camera or endoscope from the patient during the procedure and wipe off the lens with a cleaning cloth/solution. In minimally invasive surgery (MIS), the camera used for visualization within the patient is often soiled through normal operation. The debris on the lens can blur, dim or obscure patient anatomy and otherwise make it difficult to continue the operation. In manual and robotic laparoscopic procedures, it is common practice to physically remove the camera from the patient and manually wipe the lens by hand to restore clear visualization.

The traditional approach of removing the camera or the endoscope from the individual undergoing a procedure (e.g., patient) in order to clean a lens or window of said camera or said endoscope takes time and disrupts the flow of the surgical procedure. Further, in the case of surgical robotics or telemedicine, the surgeon is no longer located at the patient's bedside to physically remove the camera or endoscope in order to clean its lens which potentially makes the traditional lens cleaning approach even more disruptive to a medical or surgical procedure.

### Cleaning Lenses or Windows

Described herein is a system and method that uses the motion of the camera or endoscope within a body of an individual undergoing a medical or surgical procedure to assist in the cleaning of the lens or a window of said camera or endoscope. The systems and methods described herein include one or more mechanisms that cause a camera or endoscope to move against a stationary wiping object that is within the body of an individual so that a lens or window of the camera or endoscope is cleaned by the wiping object. A benefit of the wiping object being stationary relative to a camera or endoscope within a body of an individual undergoing a medical or surgical procedure is that there is no need to add any additional control mechanisms to actuate the wiping object for wiping of the lens or window, because it occurs within a range of motion of a camera or endoscope (i.e. when the camera or endoscope lens or window is moved against the stationary wiping object). A camera may be moved against the stationary wiping object through electrical signals, manual manipulation of a psychical mechanism, or a combination thereof.

Benefits of the systems and methods described herein generally include that surgeons will no longer need assistants to clean the camera when in the body and can clean the camera lenses with a push of a button. And as above, no complex mechanical parts are needed in addition to mechanisms that cause the movement of a camera or endoscope.

Described herein is a system for cleaning a lens or window of an imaging device used in a medical or surgical procedure while the imaging device is within a body of an individual (e.g., patient) undergoing the medical or the surgical procedure, the system comprising: (a) the imaging device comprising the lens or the window; and (b) a wiping object positioned an offset distance away from the imaging device and configured to contact at least a portion of the lens or window as the imaging device is moved along a path that uses one or more degrees of freedom of the imaging device.

Also described herein is a method for cleaning a lens or a window of an imaging device used in a medical or surgical procedure while the imaging device is within a body of an individual (e.g., patient) undergoing the medical or the surgical procedure, the method comprising: (a) providing the imaging device to be inserted in the body of the individual, wherein the imaging device is coupled to a wiping object; and (b) contacting and moving the imaging device relative to the wiping object, wherein the contact and movement cleans the lens or the window of the imaging device. In some embodiments, an imaging device may comprise a camera, an endoscope, or any other device capable of transmitting an image.

In some embodiments, a wiping object is positioned at a distal end of an imaging device near a lens or a viewing window. In some embodiments, a window is positioned between a wiping object and an imaging device. In some embodiments, a lens is positioned between a wiping object and the body of an imaging device. In some embodiments, a lens may directly come in contact with a wiping object. In some embodiments, a translucent material (e.g., glass window, plastic window, coating) may be positioned between a lens and a wiping object. In some embodiments, a lens may be positioned behind a window. In some embodiments, the imaging device includes more than one lens. In some embodiments, the imaging device includes more than one lens, wherein each lens is positioned behind a window. In some embodiments, the wiping object is a wiper, and the wiper is activated while the window or lens of the imaging device is in motion. In some embodiments, upon activation of the wiping object, an image score is determined and recorded.

An image score may be utilized to determine if the lens or window needs to be wiped one or more additional times. In some embodiments, a low image score (e.g., an image lower that is lower in value than a predetermined threshold) may indicate that the transmitted image from the imaging device is unclear or unfocused due to the presence of debris or fluid on the lens or window. In such embodiments, the system will be triggered to wipe one or more additional times. In some embodiments, a low image score may be calculated as function of a minimum threshold and an output wiping score and may be presented as a percentage of the minimum threshold (e.g., baseline measurement). For example, a metric may be a modulation transfer function (MTF) to restore the system to at least 70% of the minimum resolvability requirements. An MTF may be a measurement of contrast at a specific line pair frequency (e.g., alternating black and white lines) in different regions of the field of view. In some examples, a contrast of 20% may need to be maintained at 200 line pairs per millimeter, and in such examples, wiping may restore the metric to at least 20%.

In some embodiments, a dimension (e.g., length, width, or depth) of an imaging device may be about 1 mm to about 100 mm, about 5 mm to about 100 mm, about 5 mm to about 50 mm, or about 5 mm to about 30 mm.

In some embodiments, an imaging device may comprise a lens. A lens may comprise a circular, ovular, square, rectangular, rounded square shape. In some embodiments, a dimension (e.g., length or width) of a lens is about 1 mm to about 50 mm, about 1 mm to about 25 mm, about 2 mm to about 10 mm, or about 3 mm to about 7 mm.

In some embodiments, a lens may comprise a plurality of lenses stacked on top of each other. In some embodiments, a lens may comprise a stack of 2 lenses, 3 lenses, 4 lenses, 5 lenses, 6 lenses, 7 lenses, 8 lenses, 9 lenses, 10 lenses, 11 lenses, 12 lenses, or more. In some embodiments, the total thickness of the lens or the stack of lenses is about 0.1 mm to about 12 mm, about 1 mm to about 10 mm, or about 1 mm to about 9 mm.

In some embodiments, an imaging device may comprise a window. In some embodiments, a window may be positioned between a wiping object and a lens of the imaging device. In some embodiments, a window may comprise a circular, ovular, square, rectangular, rounded square shape. In some embodiments, the window may have a substantially flat surface, a concave surface, or a convex surface. In some embodiments, a dimension (e.g., length or width) of a window is about 1 mm to about 50 mm, about 1 mm to about 25 mm, about 1 mm to about 10 mm, or about 4 mm to about 12 mm. In some embodiments, a window may comprise a thickness of about 0.1 mm to about 6 mm, about 0.1 mm to about 3 mm, about 0.1 mm to about 1 mm, or about 0.1 mm to about 0.3 mm.

In some embodiments, an imaging device comprises a light source. In some embodiments, the light source comprises a light emitting diode (LED). In some examples, an imaging device may comprise as many light sources as lenses. In some examples, a light source may be positioned adjacent to a lens. In some embodiments, a light source may be positioned behind a window of the imaging device. In some embodiments, an imaging device may comprise a sensor. In some embodiments, the sensor detects the brightness in an area of interest imaged by the imaging device. In some embodiments, the level of brightness in the area of interest for imaging that is detected by the sensor may be used to adjust the strength of the light from the light source. In some embodiments, the imaging device may not comprise a sensor. In some embodiments, an imaging device may comprise a plurality of sensors. In some examples, an imaging device may comprise at least 1 sensor, 2 sensors, 3 sensors, 4 sensors, 5 sensors, or more. In some embodiments, a sensor may be positioned adjacent to a lens. In some embodiments, a sensor may be positioned adjacent to a light source. In some embodiments, a sensor may be positioned behind a window. In some embodiments, a lens, a light source, and/or sensor may be positioned behind a single window. In some embodiments, a lens may be positioned behind a first window and a light source and/or sensor may be positioned behind a second window. In some embodiments, each lens of an imaging device may be positioned behind a first window while each light source and/or sensor may be positioned behind a second window. For example, a binocular camera comprising two lenses and two light sources may comprise a total of four windows, where each lens and light source is positioned behind a window. In another example, a binocular camera comprising two lenses, two light sources, and at least two sensors may comprise a total of four windows, where each lens and group of light source and sensor is positioned behind a window.

In some embodiments, the window or lens of an imaging device may comprise an inflexible material. An inflexible material may comprise glass, a plastic, or a combination thereof. In some embodiments, an inflexible window or lens may allow for more wipes on the surface without comprising the properties of the material of the window or lens. In some embodiments, the window or lens of an imaging device may comprise a flexible material. A flexible material may comprise a polymer (e.g., soft plastic), cellophane, optically clear adhesive (e.g., tape), optically clear elastomer membrane (e.g., a liquid lens). In some embodiments, a flexible window or lens may allow hardened fluids (e.g., blood) to be removed through deflecting the window or lens.

In some embodiments, the window or lens of an imaging device may be coated with a material that may impart anti-reflective (AR) properties, anti-fog properties, scratch-resistance properties, oleophobicity, hydrophobicity, hydrophilicity, or a combination thereof to the surface of the window or lens. The contact angle of a droplet (e.g., water) on a hydrophobic surface or oleophobic surface may comprise an angle of at least about 90 degrees (°), 100°, 110°, 120°, 130°, 140°, 150°, 160°, 170°, 180°, or more. A fluid on a hydrophobic surface or an oleophobic may comprise decreased surface tension between the fluid and the surface of the window or lens. The contact angle of a droplet (e.g., water) on a hydrophilic surface may comprise an angle of at most about 90°, 80°, 70°, 60°, 50°, 40°, 30°, 20°, 10°, or less. A fluid on a hydrophilic surface may comprise an increased surface tension between the fluid and the surface of the window or lens than a surface without the hydrophilic treatment. A coating may be applied to the surface of a lens or window through spray methods, physical vapor deposition, chemical vapor deposition, dip coating, spin coating, or a combination thereof.

In some embodiments, a window or lens may be a part of the main body of imaging device. In some embodiments, a window or lens may be integrated into the main body of imaging device.

In some embodiments, a window or lens may be offset a distance from the main body of the imaging device. In some embodiments, impact of foreign objects with the window or lens may be minimized through fortifying the window or lens or increasing the thickness of the window or lens to increase the resistance towards breaking. In some embodiments, impact of foreign objects with the window or lens may be minimized through application of a scratch-resistant coating. A scratch-resistant coating may comprise polysiloxane (e.g., Duravue®) or any other durable material (e.g., CleanVue™ PRO). In some embodiments, impact of foreign objects with the window or lens may be minimized through application of a laminate on the window or lens. In such embodiments, shards of the window or lens may be prevented from detaching upon breaking, however, still provide visual indication of the break. In some embodiments, impact of foreign objects with the window or lens may be minimized through offsetting the main body of the imaging device to from the window or lens. The main body and window or lens may be offset a distance as described elsewhere herein.

In some embodiments, the system may comprise a wiping object. A wiping object may comprise a wiper. In some embodiments, a wiping object may be referred herein as a wiper. In some embodiments, a wiping object may comprise an elongated shape. In some embodiments, a wiping object may comprise a length of at most about the dimension (e.g., length or width) of a window or lens. In some embodiments, a dimension length of a wiping object is about 1 mm to about 50 mm, about 1 mm to about 25 mm, about 1 mm to about 15 mm, or about 5 mm to about 15 mm. In some embodiments, a wiping object may comprise a thickness of about 0.05 mm to about 6.0 mm, about 0.1 mm to about 5.0 mm, about 0.1 mm to about 2.5 mm, or about 0.5 mm to about 2.5 mm. In some embodiments, the wiping object may have dimensions of about 9mm x 3.5mm x 1.6mm. In some embodiments, the wiping object may be sized to reduce drag. In some embodiments, the wiping object may be sized to reduce friction when moving relative to the lens or the window.

In some embodiments, a wiper may comprise a polyfluoroethylene. In some embodiments, a wiper may comprise PTFE or expanded (ePTFE). In some embodiments, the wiper material may comprise a material that may increase lubriciousness, increase resistance to fluid ingress, alter the flexibility, increase resistance to sterilization methods, or a combination thereof.

In some embodiments, a wiping object may comprise a pad, a sheet, a cloth, combinations thereof, or one or more layers thereof. In some embodiments, a wiping object may comprise a circular, ovular, square, rectangular, rounded square shape. In some embodiments, a wiping object may comprise an irregular shape. In some embodiments, a wiping object may have a dimension (e.g., length or width) of about 1 mm to about 50 mm, about 1 mm to about 30 mm, about 1 mm to about 15 mm, or about 5 mm to about 15 mm. In some embodiments, a wiping object may comprise a thickness of about 0.05 mm to about 6 mm, about 0.1 mm to about 5 mm, or about 0.5 mm to about 5 mm. In some embodiments, the wiping object may comprise a material that may increase lubriciousness, increase resistance to fluid ingress, alter the flexibility, increase resistance to sterilization methods, or a combination thereof.

The wiping object may comprise a size, shape, hardness, or material that may reduce drag across the surface of the window or lens while cleaning (e.g., while the wiping object is moving relative to the imaging device). In some embodiments, an inflexible lens or window may be paired with a hard (e.g. inflexible) or soft (e.g., flexible) wiping object. In some embodiments, a flexible lens or window may be paired with a soft (e.g., flexible) wiping object.

In some embodiments, the wiping object may comprise a substantially non-porous surface. In some embodiments, the wiping object may comprise a fluoropolymer, cellulose (e.g., Sugi® sponge), silicone, polyethylene, or a combination thereof. In some embodiments, the wiping object may comprise ePTFE. In some embodiments, the wiping object may comprise one or more layers of a material. In such embodiments, the wiping object may comprise an inner layer that may comprise a material comprising low compressive modulus and an outer layer that may comprise a fluoropolymer. A material comprising a low compressive modulus may comprise a compressive modulus of at most about 10 kPa, 100 kPa, 1 MPa, 10 MPa, or 100 MPa.

In some embodiments, the wiping object may comprise a material that may increase lubriciousness. In some embodiments, the wiping object may be treated with a material that may increase lubriciousness. A material that may increase lubriciousness may comprise polytetrafluoroethylene (PTFE, or Teflon™). In some embodiments, the wiping object may comprise a textured surface configured to decrease the intermolecular forces between the wiping object and the surface of a lens or window. In some embodiments, a wiping object may comprise a superhydrophobic surface. In some embodiments, a wiping object may comprise a coating of a material that may increase lubriciousness. In some embodiments, such coatings may comprise LiquiGlide®. In some embodiments, the material for the wiping material may be chosen to reduce drag of the wiping object across the lens or the window. In some embodiments, the increase in lubriciousness of the material results in reduced friction when the wiping object and the lens or the window are moving relative to each other.

In some embodiments, the wiping object may comprise a material that may increase resistance to fluid ingress. The fluid may comprise water, irrigation, blood, or any other body fluid. In some embodiments, the wiping object may comprise a coating of a material that may increase resistance to fluid ingress. In some embodiments, a material that may increase resistance to fluid ingress may comprise a hydrophobic material (e.g., water impervious material) or an oleophobic material.

In some embodiments, the wiping object may comprise a material that increase flexibility of the wiping object. In some embodiments, the wiping object may comprise a flexible material. In some embodiments, the flexible material may mold to the surface of a lens or window when the wiping object is in contact with the lens or window. In some embodiments, the wiping object may comprise a material that may improve the surface contact of the wiping object to the surface of the lens or window. In some embodiments, the wiping object may comprise one or more materials that may increase surface contact between the wiping object and the surface of the lens or window. In some embodiments, the thickness of the wiping object may be reduced, but the contact between the wiping object and surface of the lens or window may be maintained through the wiping object comprising (e.g., coupled to) a spring or any other compliant material or device. In some embodiments, the wiping object may comprise a plurality of layers stacked on top of each other or varying compliancy or thickness. In such embodiments, variable compliance throughout the wiping object may be achieved to improve surface contact between the wiping object and the surface of the window or lens. The shape, material, size, and thickness of the wiping object may be chosen so that the interactions between the wiping object and the surface of the window or lens may be minimized. For example, the shape, material, size, and thickness of the wiping object may be tailored to minimize drag while maximizing the amount of debris or fluid that is removed while cleaning.

In some embodiments, the wiping object may comprise a material compatible with one or more sterilization methods. In some embodiments, the wiping object may comprise a material that may increase resistance to degradation during sterilization methods. A sterilization method may comprise exposing the wiping object to steam, ethylene oxide, or a combination thereof. In some embodiments, a wiping object may comprise or be coated with a material that increases hydrophobicity to increase resistance to degradation when exposed to steam or ethylene oxide. In some embodiments, a wiping object may comprise or be coating with a material that is substantially resistant to absorbing moisture. In some embodiments, a wiping object may comprise or be coated with a material that is resistant to degradation when exposed to temperatures of at least about 50 degrees Celsius (° C.), 60° C., 70° C., 80° C., 90° C., 100° C., 110° C., 120° C., 130° C., 140° C., 150° C., or more.

In some embodiments, the wiping object is for single use. In some embodiments, the wiping object may be replaceable with a new wiping object. In some embodiments, the wiping object may be used more than once. In some embodiments, the wiping object may be fixed to the system and is not replaceable.

In some embodiments, a system or method as described herein includes a camera or endoscope that is positioned relative to a wiping object within a body of an individual undergoing a medical or surgical procedure such that a movement of the camera or endoscope relative to the wiping object, while the camera or endoscope remain within the body of the individual, results in a wiping off of material from a lens or window of the camera or endoscope, thereby cleaning the lens or window. In some embodiments, moving the wiping object relative to a portion of the lens or the window comprises moving the imaging device relative to the wiping object. In some embodiments, contacting the wiping object to a portion of a lens or a window, and moving the wiping object relative to the portion of the lens or the window removes a material from at least a portion of the surface of the lens or window. In some embodiments, contacting the wiping object to a portion of a lens or a window, and moving the lens or window relative to the wiping object removes a material from at least a portion of the surface of the lens or window. In some embodiments, the wiping object may be stationary while the imaging device moves relative to the wiping object. In some embodiments, the imaging device may be stationary while the wiping object moves relative to the imaging device. In some embodiments, both the imaging device and the wiping object may move relative to each other.

In some embodiments, moving (e.g., a wiping object relative to an imaging device or an imaging device relative to a wiping object) may comprise a rotation, translation, or a combination thereof. In some embodiments, moving may comprise a back-and-forth motion from a fixed pivot point. In some embodiments, a pivot point may vary over the course of a plurality of wipes. In some embodiments, a movement may comprise a speed of about 0.1 millimeter per second (mm/s) to about 15 mm/s, about 1 mm/s to about 10 mm/s, or about 1 mm/s to about 5 mm/s. The speed of a movement (e.g., rotation, translation, or a combination thereof) may be varied over the course of a plurality of wipes. In some embodiments, moving may wipe the lens or window at least 1 times, 2 times, 3 times, 4 times, 5 times, 6 times, 7 times, 8 times, 9 times, 10 times, or more.

In some embodiments, a stationary wiping object is coupled to a camera or endoscope and positioned relative to a camera or endoscope lens or window so that when the camera or endoscope is moved against the wiping object, the lens or window is cleaned. In these embodiments, the wiping object is positioned relative to the camera or endoscope so that the wiping object does not block the lens of a camera or endoscope during normal use of the camera or endoscope during a procedure (e.g., the wiping object is offset from the imaging device). In some embodiments, while wiping, the wiping object may contact the window or lens of the imaging device. In some embodiments, while not wiping, the wiping object may be situated a distance from the imaging device. In some embodiments, the wiping object may not cover at least a portion of the lens or window of the imaging device during the medical or surgical procedure. This configuration may reduce the force required from the actuators to move the imaging device in relation to the wiping object as there will be minimal frictional force between the wiping object and the lens, or window, while they are offset from each other. In some embodiments, a wiping object and imaging device may be offset a distance of about 0.05 mm to about 6 mm, about 0.1 mm to about 5 mm, or about 0.1 mm to about 1.0 mm. In some embodiments, the camera and endoscope lens or window wipes against the stationary object in a particular direction using one or more of its degrees of freedom. In some embodiments, a wiping action may have at least one, two, three, four, five, or six degrees of freedom. In some embodiments, a wiping action has one degree of freedom. In some embodiments, a wiping action may be limited to two degrees of freedom. In some embodiments, a wiping action may be limited to three degrees of freedom. In some embodiments, a wiping action may be limited to four degrees of freedom. In some embodiments, a wiping action may be limited to five degrees of freedom. In some embodiments, a wiping action may be limited to six degrees of freedom. In some embodiments, the imaging device may move in only one direction (e.g., only downward and not upward) to wipe surface contamination on the lens or window. In some embodiments, the imaging device may rotate about an axis to wipe surface contamination on the lens or window in one direction. In some embodiments, the imaging device may rotate back and forth about an axis to wipe surface contamination on the lens or window. In some embodiments, the imaging device may rotate on two axes to clean the lens or window. In some embodiments, the imaging device may be translated to relative to the lens or window clean the lens or window. In some embodiments, the imaging device may be moved by a combination of rotation and translation relative to the lens or window to clean the lens or window. In some embodiments, the imaging device may contact a wiping object (e.g., wiper or pad), move a predetermined distance in one direction, separate from the wiping object by movement of the imaging device and/or the wiping object. In some embodiments, the imaging device may re-position back to a starting point of the wiping motion, contact the wiping object, and move an additional distance relative to the wiping object in the same direction. In some embodiments, the imaging device may contact a wiping object, move a predetermined distance in one direction, the wiping object may separate from the imaging device and re-position at a starting point of the wipe, and the imaging device may move an additional distance in the same direction.

In some embodiments, a camera or endoscope includes or is coupled to a wiping object so that movement of the camera or the endoscope results in a lens or window of the camera or the endoscope contacting the wiping object. In some embodiments, a camera or endoscope's axis of rotation is centered beneath a wiping surface of a wiping object. In some embodiments, a camera or endoscope's axis of rotation is non-centered with respect a wiping surface of a wiping object. In some embodiments, articulation allow for an angular motion of larger than 180 degrees. In some embodiments could have one rotational axis with a stationary bar located above the rotation that allows for cleaning when rotating against this surface.

In some embodiments, a wiping object is located on a static supporting structure of an imaging device and contacts the imaging device's lens or window within one or more degrees of freedom of the range of motion of the imaging device. In some embodiments, the imaging device comprises a camera. In some embodiments, the imaging device comprises an endoscope. For example, an imaging device may be configured to move in line with a supporting structure of the device and then pitch to wipe the surface against a stationary wiping pad that is located on the supporting structure of the imaging device. In some embodiments, the imaging device may have dimensions of about 13 mm diameter×40 mm long. In some embodiments, the lens may have dimensions of about 5 mm diameter×4 mm long. In some embodiments, the window may have dimensions of about 8.9 mm×6 mm×0.21 mm thick.

In some embodiments, an imaging device (e.g., camera, endoscope) may comprise at least one lens or window. In some embodiments, the imaging device may comprise one lens, two lenses, three lenses, four lenses, five lenses, six lenses, seven lenses, eight lenses, nine lenses, ten lenses, or more. In some embodiments, an imaging device (e.g., a camera) is a binocular camera having two lenses and some of these embodiments, once a first lens or window of the camera is sufficiently cleaned the camera can rotate 180 degrees and begin the wiping process on the second lens or window with the wiping material making contact in the reverse direction. In some embodiments, a wipe may be in the opposite direction on one lens or window in order to maintain the same wiping motion of the wiper. In some embodiments, the wiping object is configured to contact at least a portion of a first lens or a first window and at least a portion of a second lens or a second window sequentially. In some embodiments, the system may comprise an additional wiping object (e.g., a second wiping object). In such embodiments, a first wiping object is configured to contact at least a portion of the first lens or first window and the additional wiping object is configured to contact at least a portion of the second lens or second window. In some embodiments, the first wiping object and the second wiping object contact the first lens or the first window and the second lens or the second window simultaneously.

In some embodiments, a system comprising the wiping object may be a stationary system within a surgical instrument. In some embodiments, the surgical system includes a trocar and a surgical instrument. In such embodiments, actuation of the wiped object (because wiper is not moving) may comprise at least partially moving the imaging device such that is positioned within the trocar. In such embodiments, movement (e.g., translation, rotation, or a combination thereof) of the imaging device may contact the lens or window with the wiping object, thereby cleaning the lens of window. In some embodiments, a separate instrument may be inserted into the body of an individual undergoing the medical or surgical procedure through a same or different port. In such embodiments, the surgical instrument may attach to the main body of the imaging device or move relative to the lens or window, thereby cleaning the lens or window.

A wiping object as described herein comprises a material that is suitable for removing a material (e.g. dirt, blood, adipose tissue, body fluid, irrigation, or condensation) from a lens or window of a camera or an endoscope when the wiping object contacts the lens or window. Wiping objects as described herein may be formed as wipers, pads, cloths, or sheets and may be made up of any material that is suitable for cleaning a surface include hard and soft materials as well as absorbent and non-absorbent materials and various combinations thereof. In some embodiments, a stationary wiping object may comprise ePTFE. ePTFE provides a biologically safe low coefficient non-porous surface with adjustable pliability and shapes that can be configured to have a relatively low profile.

In some embodiments, a coating can be applied to the substrate of a lens or window to prevent large build up. In some embodiments, the lens or window will have both a hydrophobic and oleophobic coating with the addition of an anti-fog coating for increased performance of the wiping object.

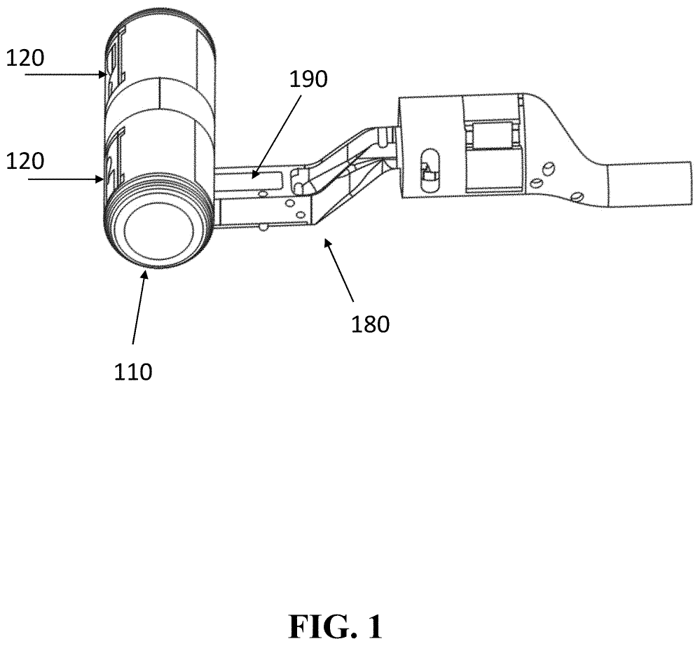

In some examples, FIG. 1 shows a camera 110 in a front facing configuration along with a wiping object 190. A wiping object 190, which in some embodiments comprises ePTFE, is mounted to the main body 180. In some embodiments, a lens of a camera may be behind a window 120 conformed to the profile of the rotation surface (not shown) so that as the camera is rotated, a part of its surface remains tangent to the wiping object 190.

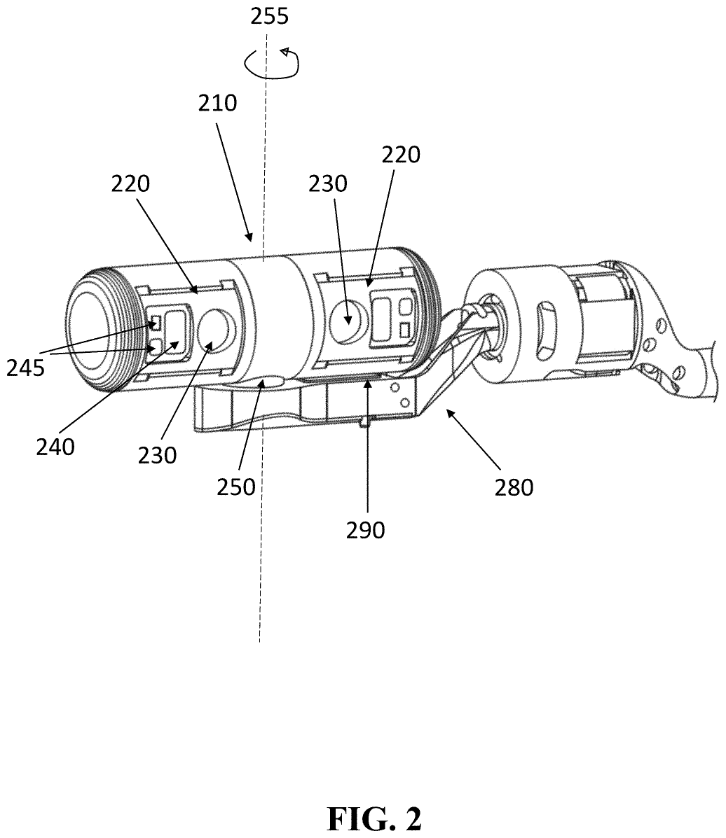

In some examples, FIG. 2 shows a camera 210 in an orthogonal direction to insertion. This maneuver is performed first using a point of rotation 250 to rotate the camera about an axis of rotation 255 in order to get the camera 210 aligned with the wiping object 290 on the main body 280. In some embodiments, one wiping object 290 surfaces both lenses 230 behind a window 220 of a binocular camera 210 by doing one at a time. In some embodiments, translation towards a second window or lens may not needed if two wiping objects were to be used. In some embodiments, a camera 210 may comprise a light source comprising a light emitting diode (LED) 240 and/or one or more sensors 245 behind a window 220.

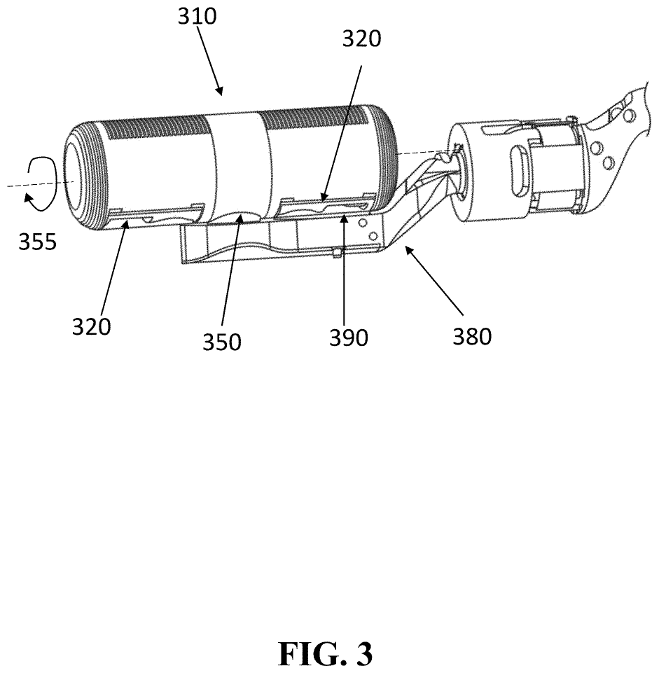

In some embodiments, FIG. 3 shows a camera 310 pointed downward while coupled to the main body 380. The camera may move from the position as shown in FIG. 2 past the position shown in FIG. 3 so that the entire surface of the window 320 or lens (not shown) is cleaned. Multiple wipes using the wiping object 390 might need to be performed. In some embodiments, multiple wipes may occur through rolling the camera 310 along the rotation point 350 about an axis of rotation 355.

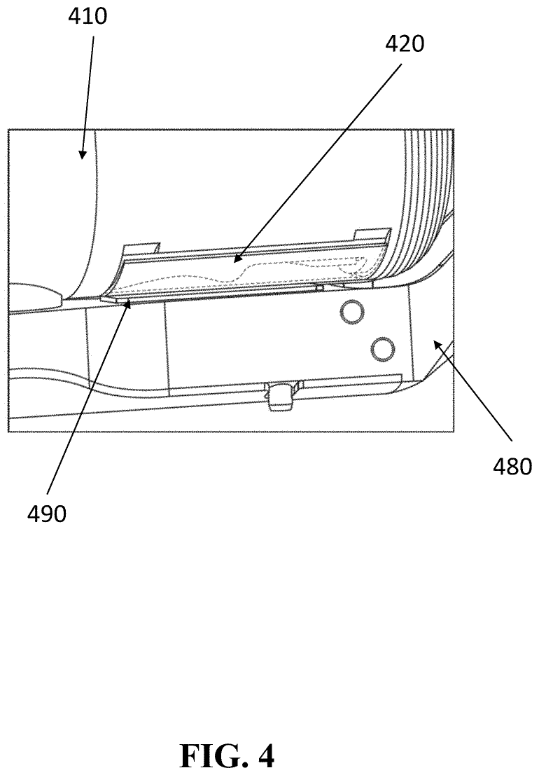

In some examples, FIG. 4 shows a close up of a lens (not shown) behind a window 420 and a wiping object 490 which in some embodiments comprises ePTFE. In this case, the ePTFE conforms to the surface. In some embodiments, materials such as silicon may conform like that of a car windshield wiper be used to increase force on the lens or window while minimally affecting the movement quality.

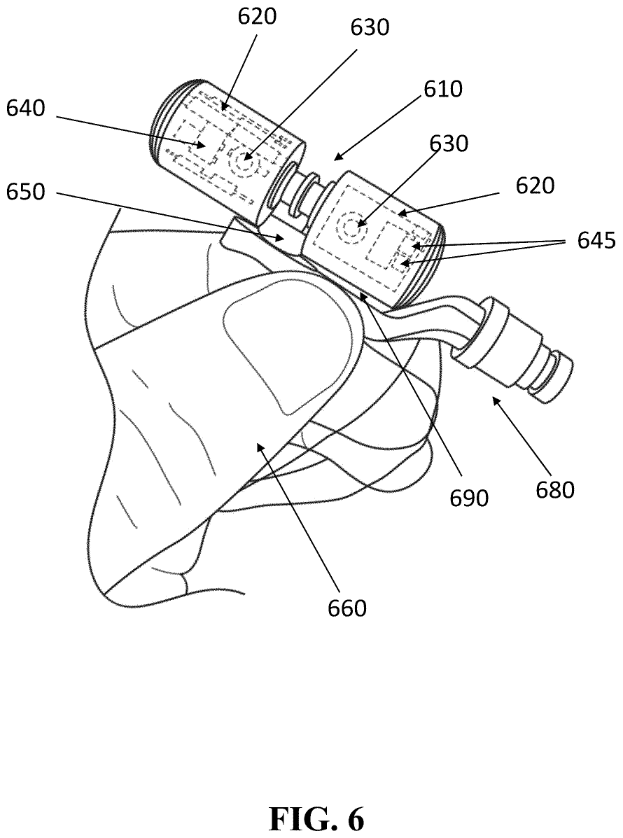

In some examples, FIG. 6 shows a camera 610 coupled to a main body 680 with an automated wiping object 680 built in. As shown, the wiping object 680 is located above the thumb 660 shown in the figure. The camera may 610 comprise two lenses 630, an light source comprising a LED 640, and one or more sensors 645 positioned behind separate windows 620. In this embodiment, the camera 610 has been rotated about the point of rotation 650 so that a window 620 and lens 630 will be aligned with the wiping object 690 upon rotation in a separate direction.

### Automated Initiation of Lens Cleaning

Described herein are devices, systems, and methods for initiating automated cleaning of a lens or window of an imaging device during a medical or surgical procedure, comprising: (a) providing an eye tracking device, wherein the eye tracking device comprises an eye tracking hardware; (b) coupling the eye tracking device with at least one eye of an individual performing the medical or the surgical procedure; (c) providing the imaging device to be inserted in the body of the individual, wherein the imaging device is coupled to a wiping object; (d) tracking movements of the individual; and (e) detecting an activating movement among the movements to trigger the imaging device to contract and move relative to a wiping object, wherein the contact and movement cleans the lens or window of the imaging device.

A benefit of this system may comprise the ability of the imaging device to remain within the surgical space while the cleaning procedure occurs, while simultaneously affording the individual performing the medical or surgical procedure the ability to initiate cleaning.

Described herein is an eye tracking device fitted to track the eyes of the surgeon whereby blinking or doing some other specific motion with the eyes is utilized to trigger a fast, automated lens or window cleaning procedure. In some embodiments, initiation of the cleaning procedure may occur as an eye tracking device observing direct signals from the individual performing the procedure. In some embodiments, a movement may comprise movement of the eye. The surgeon's eyes are tracked by eye tracking hardware known in the art such as foveal tracking, computer vision (CV), electrical potential measurement, contact lenses, etc. In some embodiments, an eye tracking device may be worn on the head of an individual. In some embodiments, an eye tracking device may be positioned a distance from the individual, a camera may track a movement. In some embodiments, an eye tracking device may be positioned within a stationary viewfinder (e.g., a stereoscopic microscope). A signal may comprise a sharp look in a specified direction (e.g., towards a camera coupled to the eye tracking device), focusing on a target (e.g., object or location) in space, one or more blinks, one or more long blinks, one or more winks, one or more long winks, eye strain, a large head motion away from a video output display, or combinations thereof. In some embodiments, one or more blinks or winks may last for about 0.01 seconds (s) to about 5 s, about 0.05 s to about 3 s, or about 0.1 s to about ls. In some embodiments, an eye movement may comprise focusing on a target object or location in space for a period of time. A period of time may be about 0.1 seconds (s) to about 20 s, about 0.5 s to about 10 s, about 0.5 s to about 5 s, or about 1 s to about 5 s. In some embodiments, a movement of eyes may comprise looking in a first direction followed by looking in a second direction within a given timeframe. In such embodiments, a timeframe may be about 0.1 seconds (s) to 20 s, about 0.5 s to about 10 s, about 0.5 s to about 5 s, or about 1 s to about 5 s. In some embodiments, the surgeon wears a head mounted display fitted with eye tracking. In some embodiments, the surgeon views a display mounted some distance from their eyes and their eyes are tracked via a camera. In some embodiments the surgeon views through a stationary viewfinder (like a stereoscopic microscope) fitted with eye tracking.

In some embodiments, an individual performing the procedure may be fitted with a head tracking system. In such examples, the head tracking system may track the individual's head movements (e.g., head shaking) using tracking hardware known in the art. A head tracking system may comprise a 3D display, a virtual reality (VR) headset, a vision engineering system, or any other system known in the art. In some embodiments, a hand controller or foot controller may be used to trigger a wiping motion. In some embodiments, a wiping motion may be triggered through a voice command. In some embodiments, a wiping motion may be triggered through touching a specific part of the computer system.

In one embodiment, the system detects when the surgeon blinks and initiates the lens or window cleaning process. Due to the frequency that humans blink, this may result in many unnecessary clean processes to be initiated. To those ends, in some embodiments, the surgeon must blink or close their eyes for a specified period of time to trigger the cleaning process. For example, a surgeon may blink or close their eyes for about 1 second to trigger the cleaning process. In some embodiments, the cleaning process is triggered by the surgeon focusing on a specific object, target, or location in space for a period of time. For example it could be triggered by the surgeon looking up for 0.5 s. In some embodiments, it is triggered by a specific motion of the eyes, for example, looking left and then looking right within 0.5 s.

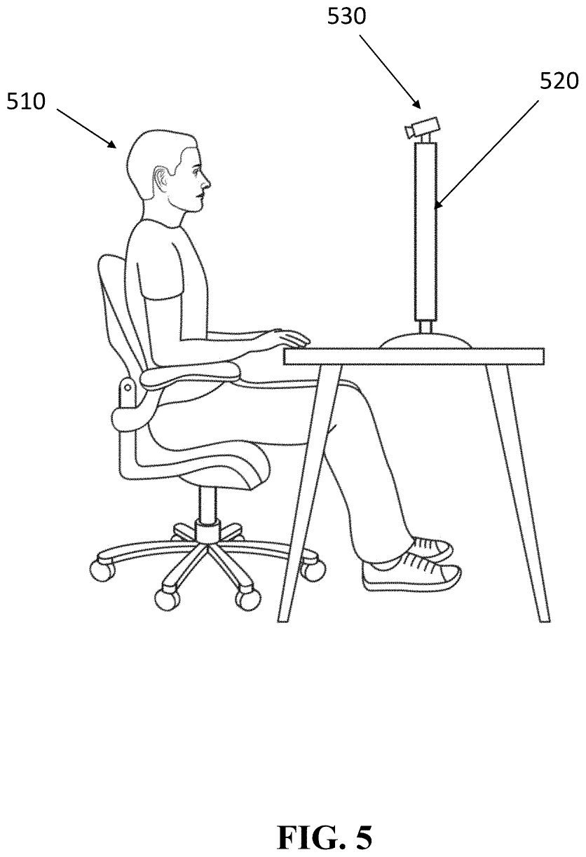

FIG. 5 shows a surgeon 510 sitting at a control console 520 operating a surgical robot. There is an eye tracking camera 530 mounted above the display that is used to control when the lens or window cleaning process starts.

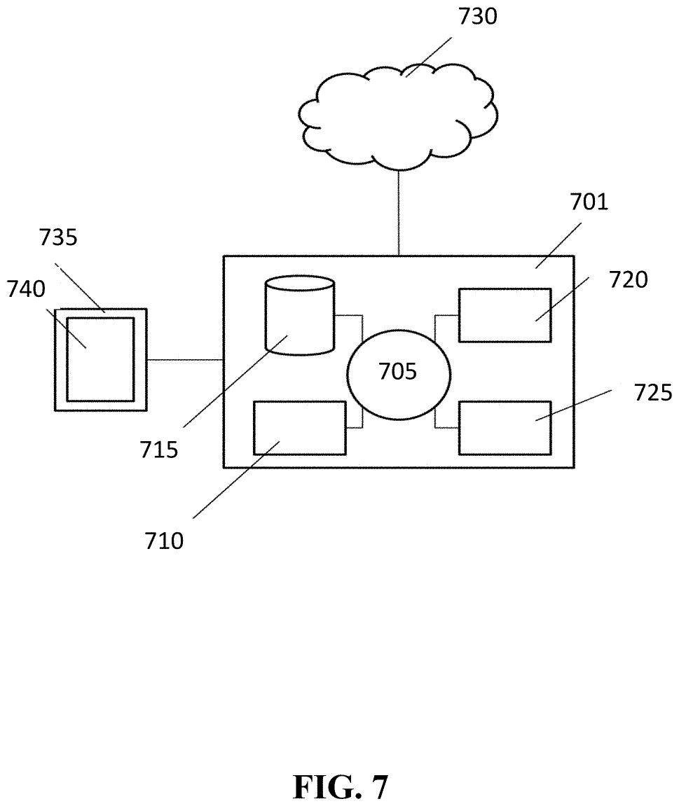

FIG. 7 shows one example of a computer system 701 that is programmed or configured to direct operation of a device or system as described herein, including imaging, cleaning the lens or window, tracking the movement of a user of the system, and activating the cleaning of the lens or window. The computer system 701 regulates various aspects of systems and methods of the present disclosure, such as, (a) movement of one or more device or system components, (b) operation of one or more device or system components (e.g., one or more positioning elements, one or more wrist elements, and/or one or more cameras) (c) adjustment of one or more parameters of one or more device or system components, (d) computational evaluation of one or more measurements of a device or system, and (e) display of various parameters including input parameters, results of a measurement, or any combination of any of these.

In some embodiments, a computer system 701 is an electronic device of a user (e.g. smartphone, laptop, computer, headset, wrist device). In some embodiments, the computer system 701 is remotely located with respect to the devices or systems provided herein. The electronic device, in some embodiments, is a mobile electronic device.

The computer system 701 includes a central processing unit (CPU, also “processor” and “computer processor” herein) 705, which, in some embodiments, is a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 1210, storage unit 715, interface 720 and peripheral devices 725 are in communication with the CPU 705 through a communication bus (solid lines), such as a motherboard. The storage unit 715 is configured as a data storage unit (or data repository) for storing data. The computer system 701 is operatively coupled to a computer network (“network”) 730 with the aid of the communication interface 720. The network 730 is the Internet, an interne and/or extranet, or an intranet and/or extranet that is in communication with the Internet. The network 730 in some embodiments is a telecommunication and/or data network. The network 730 includes one or more computer servers, which enable distributed computing, such as cloud computing. The network 730, in some embodiments, with the aid of the computer system 701, implements a peer-to-peer network, which enables devices coupled to the computer system 701 to behave as a client or a server.

The CPU 705 is configured to execute a sequence of machine-readable instructions, which may be embodied in a program or software. The instructions are stored in a memory location, such as the memory 710. The instructions are directed to the CPU 705, which subsequently program or configure the CPU 705 to implement methods provided herein. Examples of operations performed by the CPU 705 include fetch, decode, execute, and writeback.

The CPU 705 is part of a circuit, such as an integrated circuit. One or more other components of the system 701 are included in the circuit. In some embodiments, the circuit is an application specific integrated circuit (ASIC).

The storage unit 715 stores files, such as drivers, libraries and saved programs. The storage unit 715 stores user data, e.g., user preferences and user programs. The computer system 701 in some embodiments include one or more additional data storage units that are external to the computer system 701, such as located on a remote server that is in communication with the computer system 701 through an intranet or the Internet.

The computer system 701 communicates with one or more remote computer systems through the network 730. For instance, the computer system 701 communicates with a remote computer system of a user (e.g., a second computer system, a server, a smart phone, an iPad, or any combination thereof). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device), or personal digital assistants. The user accesses the computer system 701 via the network 730.

Methods as described herein are implemented by way of machine (e.g., computer processor) executable code stored on an electronic storage location of the computer system 701, such as, for example, on the memory 710 or electronic storage unit 715. The machine executable or machine readable code is provided in the form of software. During use, the code is executed by the processor 705. In some cases, the code is retrieved from the storage unit 715 and stored on the memory 710 for ready access by the processor 705. In some cases, the electronic storage unit 715 is precluded, and machine-executable instructions are stored on memory 710.

A machine readable medium, such as computer-executable code, takes many forms, including but not limited to, a tangible storage medium, a carrier wave medium or physical transmission medium. Non-volatile storage media include, for example, optical or magnetic disks, such as any of the storage devices in any computer(s) or the like, such as is used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media takes the form of electric or electromagnetic signals, or acoustic or light waves such as those generated during radio frequency (RF) and infrared (IR) data communications. Common forms of computer-readable media include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer reads programming code and/or data. Many of these forms of computer readable media is involved in carrying one or more sequences of one or more instructions to a processor for execution.

The computer system 701, in some embodiments, includes or is in communication with an electronic display 735 that comprises a user interface (UI) 740 for providing, for example, a graphical representation or other visualization (e.g., image data or video data) of operation of the robotic arm during surgery, one or more parameters that are input or adjusted by a user or by a controller, or any combination thereof. Examples of UIs include, without limitation, a graphical user interface (GUI) and web-based user interface.

Methods and systems provided herein may be implemented by one or more algorithms. An algorithm, in some embodiments, is implemented by way of software upon execution by the central processing unit 705. The algorithm may, for example, regulate various aspects of systems and methods of the present disclosure, such as, for example, (a) movement of one or more device or system components, (b) operation of one or more parameters of one or more device or system components (c) adjustment of one or more parameters of one or more device or system components, (d) computational evaluation of one or more measurements of a device or system, and (e) display of various parameters including input parameters, results of a measurement, or any combination of any of these.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “about,” as used herein, means within ±10% of a value. For example, if it is stated, “a lens comprises a thickness of about 10 mm”, it is implied that the thickness may be from 9 mm to 11 mm.

The term “comprise” and variations thereof, such as, “comprises” and “comprising” are to be construed in an open, inclusive sense, that is, as “including, but not limited to.”

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The term “optional” or “optionally” denotes that a subsequently described event or circumstance can but need not occur, and that the description includes instances where the event or circumstance occurs and instances in which it does not.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXEMPLARY EMBODIMENTS

Among the exemplary embodiments are:

Embodiment 1 comprises a system for cleaning a lens or window of an imaging device during a surgical procedure within a patient, the system comprising: the imaging device comprising the lens or the window; and a wiping object positioned an offset distance away from the imaging device and configured to contact at least a portion of the lens or the window as the imaging device is moved along a path that uses one or more degrees of freedom of the imaging device. Embodiment 2 comprises the system of embodiment 1, wherein the imaging device comprises a camera or an endoscope. Embodiment 3 comprises the system of embodiment 1, wherein contacting the wiping object to the portion of the lens or the window and moving the wiping object relative to the portion of the lens removes a material from at least a portion of a surface of the lens or the window. Embodiment 4 comprises the system of embodiment 3, wherein the material comprises blood, adipose tissue, body fluid, irrigation, condensation, or a combination thereof. Embodiment 5 comprises the system of embodiment 3, wherein the moving of the wiping object relative to the portion of the lens or the window comprises moving the imaging device relative to the wiping object. Embodiment 6 comprises the system of embodiment 5, wherein the moving of the imaging device comprises rotation, translation, or a combination thereof. Embodiment 7 comprises he system of embodiment 5, wherein the moving of the wiping object relative to the portion of the lens or the window comprises the wiping object being stationary. Embodiment 8 comprises the system of embodiment 3, wherein the moving of the wiping object relative to the portion of the lens or the window comprises moving the wiping object relative to the imaging device. Embodiment 9 comprises the system of embodiment 3, wherein the imaging device moves relative to the wiping object at a speed of about 0.5 mm/s to about 10.0 mm/s. Embodiment 10 comprises the system of embodiment 1, wherein the wiping object and the imaging device are not in contact when the lens or the window cleaning is not occurring. Embodiment 11 comprises the system of embodiment 10, wherein the body of the imaging device is positioned a distance from the wiping object. Embodiment 12 comprises the system of embodiment 11, wherein the distance is at least about 0.1 mm to about 1.0 mm. Embodiment 13 comprises the system of embodiment 1, wherein the wiping object does not cover at least a portion of the lens or the window of the imaging device during the medical or the surgical procedure. Embodiment 14 comprises the system of embodiment 1, wherein the imaging device comprises a first lens and a second lens, wherein the first lens is positioned behind a first window and the second lens is positioned behind a second window. Embodiment 15 comprises the system of embodiment 14, wherein the wiping object is configured to contact at least a portion of the first window and at least a portion of the second window sequentially. Embodiment 16 comprises the system of embodiment 14, wherein the system comprises an additional wiping object, wherein the wiping object is configured to contact at least a portion of the first window and the additional wiping object is configured to contact at least a portion of the second window. Embodiment 17 comprises the system of embodiment 16, wherein the wiping object is configured to contact at least a portion of the first window and the additional wiping object is configured to contact at least a portion of the second window simultaneously. Embodiment 18 comprises the system of embodiment 1, wherein the wiping object comprises a pad, a sheet, or a wiper, or a combination thereof. Embodiment 19 comprises the system of embodiment 1, wherein the wiping object is sized to reduce drag across the surface of the lens or the window when the wiping object is moved relative to the lens. Embodiment 20 comprises the system of embodiment 19, wherein the wiping object comprises a material treated to increase lubriciousness. Embodiment 21 comprises the system of embodiment 19, wherein the wiping object comprises a material treated to increase resistance to fluid ingress. Embodiment 22 comprises the system of embodiment 1, wherein the wiping object comprises a flexible material. Embodiment 23 comprises the system of embodiment 22, wherein the flexible material molds to the surface of the lens or the window when the wiping object is in contact with the lens or the window. Embodiment 24 comprises the system of embodiment 22, wherein the wiping object comprises a material to improve surface contact to the surface of the lens or the window. Embodiment 25 comprises the system of embodiment 1, wherein the wiping object comprises a material compatible with one or more sterilization methods. Embodiment 26 comprises the system of embodiment 1, wherein the wiping object comprises a substantially non-porous surface. Embodiment 27 comprises the system of embodiment 1, wherein the wiping object comprises a fluoropolymer. Embodiment 28 comprises the system of embodiment 27, wherein the fluoropolymer comprises expanded polytetrafluoroethylene (ePTFE). Embodiment 29 comprises the system of embodiment 28, wherein the wiping object comprises one or more layers of ePTFE. Embodiment 30 comprises the system of embodiment 29, wherein the wiping object comprises an inner layer comprising a material having a low compressive modulus and an outer layer of fluoropolymer. Embodiment 31 comprises the system of embodiment 1, wherein the lens comprises a stack of two or more lenses. Embodiment 32 comprises the system of embodiment 31, wherein the stack of lenses is about 1 mm thick to about 10 mm thick. Embodiment 33 comprises the system of embodiment 1, wherein the lens or the window comprises glass, plastic, or a combination thereof. Embodiment 34 comprises the system of embodiment 33, wherein the lens or the window comprises a flexible material. Embodiment 35 comprises the system of embodiment 33, wherein the lens or the window comprises a coating. Embodiment 36 comprises the system of embodiment 35, wherein the coating laminates the lens or the window. Embodiment 37 comprises the system of embodiment 35, wherein the coating provides an increase in hydrophobicity, hydrophilicity, oleophilicity, anti-fogging, anti-reflection, scratch-resistance, or a combination thereof. Embodiment 38 comprises the system of embodiment 35, wherein the coating is applied to the lens or the window through spraying, spin coating, chemical vapor deposition, physical vapor deposition, or dipping.

Embodiment 39 comprises a method for cleaning a lens or a window of an imaging device during a medical or surgical procedure in a body of an individual, the method comprising: (a) providing the imaging device to be inserted in the body of the individual, wherein the imaging device is coupled to a wiping object; and (b) contacting and moving the imaging device relative to the wiping object, wherein the contact and movement cleans the lens or the window of the imaging device. Embodiment 40 comprises the method of embodiment 39, wherein the imaging device comprises a camera or an endoscope. Embodiment 41 comprises the method of embodiment 39, wherein contacting the wiping object to the portion of the lens or the window and moving the wiping object relative to the portion of the lens removes a material from at least a portion of a surface of the lens. Embodiment 42 comprises the method of embodiment 41, wherein the material comprises blood, adipose tissue, body fluid, irrigation, condensation, or a combination thereof. Embodiment 43 comprises the method of embodiment 41, wherein the moving of the wiping object relative to the portion of the lens or the window comprises moving the imaging device relative to the wiping object. Embodiment 44 comprises the method of embodiment 43, wherein the moving of the imaging device comprises rotation, translation, or a combination thereof. Embodiment 45 comprises the method of embodiment 43, wherein the moving of the wiping object relative to the portion of the lens or the window comprises the wiping object being stationary. Embodiment 46 comprises the method of embodiment 41, wherein the moving of the wiping object relative to the portion of the lens or the window comprises moving the wiping object relative to the imaging device. Embodiment 47 comprises the method of embodiment 41, wherein the imaging device moves relative to the wiping object at a speed of about 0.5 mm/s to about 2.0 mm/s. Embodiment 48 comprises the method of embodiment 39, wherein the wiping object and the imaging device are not in contact when lens or the window cleaning is not occurring. Embodiment 49 comprises the method of embodiment 48, wherein the body of the imaging device is positioned a distance from the wiping object. Embodiment 50 comprises the method of embodiment 49, wherein the distance is at least about 0.1 mm to about 1.0 mm. Embodiment 51 comprises the method of embodiment 39, wherein the wiping object does not cover at least a portion of the lens or the window of the imaging device during the medical or the surgical procedure to allow for visualization by the imaging device. Embodiment 52 comprises the method of embodiment 39, wherein the imaging device comprises a first lens and a second lens, wherein the first lens is positioned behind a first window and the second lens is positioned behind a second window. Embodiment 53 comprises the method of embodiment 52, wherein the wiping object is configured to contact at least a portion of the first window and at least a portion of the second window sequentially. Embodiment 54 comprises the method of embodiment 52, wherein the system comprises an additional wiping object, wherein the wiping object is configured to contact at least a portion of the first window and the additional wiping object is configured to contact at least a portion of the second window. Embodiment 55 comprises the method of embodiment 54, wherein the wiping object is configured to contact at least a portion of the first window and the additional wiping object is configured to contact at least a portion of the second window simultaneously. Embodiment 56 comprises the method of embodiment 39, wherein the wiping object comprises a pad, a sheet, or a wiper, or a combination thereof. Embodiment 57 comprises the method of embodiment 39, wherein the wiping object is sized to reduce drag across the surface of the lens or the window when the wiping object is moved relative to the lens or the window. Embodiment 58 comprises the method of embodiment 57, wherein the wiping object comprises a material treated to increase lubriciousness. Embodiment 59 comprises the method of embodiment 57, wherein the wiping object comprises a material treated to increase resistance to fluid ingress. Embodiment 60 comprises the method of embodiment 39, wherein the wiping object comprises a flexible material. Embodiment 61 comprises the method of embodiment 60, wherein the flexible material molds to the surface of the lens or the window when the wiping object is in contact with the lens or the window. Embodiment 62 comprises the method of embodiment 60, wherein the wiping object comprises a material to improve surface contact to the surface of the lens or the window. Embodiment 63 comprises the method of embodiment 39, wherein the wiping object comprises a material compatible with one or more sterilization methods. Embodiment 64 comprises the method of embodiment 39, wherein the wiping object comprises a substantially non-porous surface. Embodiment 65 comprises the method of embodiment 39, wherein the wiping object comprises a fluoropolymer. Embodiment 66 comprises the method of embodiment 65, wherein the fluoropolymer comprises expanded polytetrafluoroethylene (ePTFE). Embodiment 67 comprises the method of embodiment 65, wherein the wiping object comprises one or more layers of ePTFE. Embodiment 68 comprises the method of embodiment 65, wherein the wiping object comprises an inner layer comprising a material having a low compressive modulus and an outer layer of fluoropolymer. Embodiment 69 comprises the method of embodiment 39, wherein the lens comprises a stack of two or more lenses. Embodiment 70 comprises the method of embodiment 69, wherein the stack of two or more lenses is about 1 mm thick to about 10 mm thick. Embodiment 71 comprises the method of embodiment 39, wherein the lens or the window comprises glass, plastic, or a combination thereof. Embodiment 72 comprises the method of embodiment 71, wherein the lens or the window comprises a flexible material. Embodiment 73 comprises the method of embodiment 71, wherein the lens or the window comprises a coating. Embodiment 74 comprises the method of embodiment 73, wherein the coating laminates the lens or the window. Embodiment 75 comprises the method of embodiment 73, wherein the coating provides an increase in hydrophobicity, hydrophilicity, oleophilicity, anti-fogging, anti-reflection, scratch-resistance, or a combination thereof. Embodiment 76 comprises the method of embodiment 73, wherein the coating is applied to the lens or the window through spraying, spin coating, chemical vapor deposition, physical vapor deposition, or dipping. Embodiment 77 comprises the method of embodiment 39, wherein the moving the imaging device relative to the wiping object wipes the lens or the window at least two times. Embodiment 78 comprises the method of embodiment 77, wherein the moving imaging device relative to the wiping object wipes the lens or the window at least 5 times. Embodiment 79 comprises the method of embodiment 39, wherein the method further comprises, subsequent to (b), recording an image score.

Embodiment 80 comprises a method for initiating automated cleaning of a lens or a window of an imaging device during a medical or surgical procedure, the method comprising: (a) providing an eye tracking device, wherein the eye tracking device comprises an eye tracking hardware; (b) coupling the eye tracking device with at least one eye of an individual performing the medical or the surgical procedure; (c) providing the imaging device to be inserted in the body of the individual, wherein the imaging device is coupled to a wiping object; (d) tracking movements of the individual; and (e) detecting an activating movement among the movements to trigger the imaging device to contract and move relative to a wiping object, wherein the contact and movement cleans the lens or the window of the imaging device. Embodiment 81 comprises the method of embodiment 80, wherein the movement comprises movement of the eye. Embodiment 82 comprises the method of embodiment 81, wherein the movement of the eye comprises blinking. Embodiment 83 comprises the method of embodiment 82, wherein the blinking comprises one blink, wherein the one blink lasts for about 0.3 seconds. Embodiment 84 comprises the method of embodiment 82, wherein the movement of the eye comprises focusing on a target object or location in space for a period of time. Embodiment 85 comprises the method of embodiment 84, wherein the period of time is about 2 seconds. Embodiment 86 comprises the method of embodiment 82, wherein the movement of the eye comprises looking in a first direction followed by looking in a second direction. Embodiment 87 comprises the method of embodiment 86, wherein the looking in the first direction followed by looking in the second direction occurs within about 1 second. Embodiment 88 comprises the method of embodiment 82, wherein the movement of the eye comprises a sharp look in a specified direction. Embodiment 89 comprises the method of embodiment 88, wherein the specified direction is towards a camera coupled to the eye tracking device. Embodiment 90 comprises the method of embodiment 80, wherein the eye tracking hardware comprises foveal tracking, computer vision, electrical potential measurement, contact lenses, or a combination thereof. Embodiment 91 comprises the method of embodiment 90, wherein the eye tracking hardware comprises foveal tracking. Embodiment 92 comprises the method of embodiment 80, wherein the eye tracking device is worn on a head of the individual. Embodiment 93 comprises the method of embodiment 80, wherein the eye tracking device is positioned a distance from the individual, and wherein a camera tracks the at least one movement. Embodiment 94 comprises the method of embodiment 80, wherein the eye tracking device is positioned within a stationary viewfinder. Embodiment 95 comprises the method of embodiment 94, wherein the stationary viewfinder is a stereoscopic microscope.

What is claimed is:

1. A system for cleaning a lens or window of an imaging device during a surgical procedure within a patient, the system comprising:
the imaging device comprising the lens or the window; and
a wiping object positioned an offset distance away from the imaging device and configured to contact at least a portion of the lens or the window as the imaging device is moved along a path that uses one or more degrees of freedom of the imaging device.

2. The system of claim 1, wherein the imaging device comprises a camera or an endoscope.

3. The system of claim 1, wherein contacting the wiping object to the portion of the lens or the window and moving the wiping object relative to the portion of the lens removes a material from at least a portion of a surface of the lens or the window.

4. The system of claim 3, wherein the moving of the wiping object relative to the portion of the lens or the window comprises moving the imaging device relative to the wiping object.

5. The system of claim 4, wherein the moving of the imaging device comprises rotation, translation, or a combination thereof.

6. The system of claim 4, wherein the moving of the wiping object relative to the portion of the lens or the window comprises the wiping object being stationary.

7. The system of claim 3, wherein the moving of the wiping object relative to the portion of the lens or the window comprises moving the wiping object relative to the imaging device.

8. The system of claim 1, wherein the wiping object and the imaging device are not in contact when the lens or the window cleaning is not occurring.

9. The system of claim 1, wherein the imaging device comprises a first lens and a second lens, wherein the first lens is positioned behind a first window and the second lens is positioned behind a second window.

10. The system of claim 9, wherein the wiping object is configured to contact at least a portion of the first window and at least a portion of the second window sequentially.

11. The system of claim 9, wherein the system comprises an additional wiping object, wherein the wiping object is configured to contact at least a portion of the first window and the additional wiping object is configured to contact at least a portion of the second window.

12. The system of claim 1, wherein the wiping object comprises a pad, a sheet, or a wiper, or a combination thereof.

13. The system of claim 1, wherein the wiping object is sized to reduce drag across the surface of the lens or the window when the wiping object is moved relative to the lens.

14. The system of claim 1, wherein the wiping object comprises a flexible material configured to mold to the surface of the lens or the window when the wiping object is in contact with the lens or the window.

15. The system of claim 1, wherein the wiping object comprises a substantially non-porous surface.

16. The system of claim 1, wherein the wiping object comprises a fluoropolymer.

17. A method for cleaning a lens or a window of an imaging device during a medical or surgical procedure in a body of an individual, the method comprising:
(a) providing the imaging device to be inserted in the body of the individual, wherein the imaging device is coupled to a wiping object; and
(b) contacting and moving the imaging device relative to the wiping object, wherein the contact and movement cleans the lens or the window of the imaging device.

18. The method of claim 17, wherein the imaging device comprises a camera or an endoscope.

19. The method of claim 17, wherein contacting the wiping object to the portion of the lens or the window and moving the wiping object relative to the portion of the lens removes a material from at least a portion of a surface of the lens.

20. The method of claim 19, wherein the material comprises blood, adipose tissue, body fluid, irrigation, condensation, or a combination thereof.

21. The method of claim 19, wherein the moving of the wiping object relative to the portion of the lens or the window comprises moving the imaging device relative to the wiping object.

22. The method of claim 21, wherein the moving of the imaging device comprises rotation, translation, or a combination thereof.

23. The method of claim 21, wherein the moving of the wiping object relative to the portion of the lens or the window comprises the wiping object being stationary.

24. The method of claim 19, wherein the moving of the wiping object relative to the portion of the lens or the window comprises moving the wiping object relative to the imaging device.

25. The method of claim 17, wherein the wiping object and the imaging device are not in contact when lens or the window cleaning is not occurring.

26. The method of claim 17, wherein the imaging device comprises a first lens and a second lens, wherein the first lens is positioned behind a first window and the second lens is positioned behind a second window.

27. The method of claim 26, wherein the wiping object is configured to contact at least a portion of the first window and at least a portion of the second window sequentially.

28. The method of claim 26, wherein the system comprises an additional wiping object, wherein the wiping object is configured to contact at least a portion of the first window and the additional wiping object is configured to contact at least a portion of the second window.

29. The method of claim 17, wherein the moving the imaging device relative to the wiping object wipes the lens or the window at least two times.
